(19) United States
(12) Patent Application Publication
(10) Pub. No.: US 2024/0373946 A1
(43) Pub. Date: Nov. 14, 2024

(54) Inhalation Device and Method of Manufacturing an Inhalation Device

(71) Applicant: JT International SA, Geneva (CH)

(72) Inventor: Akira Yamaguchi, Geneva (CH)

(73) Assignee: JT International SA, Geneva (CH)

(21) Appl. No.: 18/683,125

(22) PCT Filed: Aug. 9, 2022
(86) PCT No.: PCT/EP2022/072332

§ 371 Date: Feb. 12, 2024

(30) Foreign Application Priority Data
Aug. 13, 2021 (EP) 21191267.0

(51) Int. Cl. A24F 40/70; A61M 15/00

(52) U.S. Cl. CPC A24F 40/70; A61M 15/0086; A61M 2205/0211

(57) ABSTRACT

An inhalation device has a housing at least partially made of a hard material and at least one button provided in an opening of the housing and at least partially made of a material substantially as hard as that of the housing or harder, wherein the button is attached to the housing by an elastic material on the inside of the housing. A method of producing an inhalation device includes the steps of providing a housing at least partially made of a hard material and providing at least one button at least partially made of a material substantially as hard as that of the housing or harder and attaching at least one button in an opening of the housing by an elastic material attached to the inside of the housing.

### TECHNICAL FIELD

The invention relates to an inhalation device and a method of manufacturing an inhalation device.

In recent years, conventional smoking products have been more and more replaced by aerosol-generating devices, in which a liquid is evaporated and can be inhaled by a user. In this context, it is desirable, to use relatively hard materials for as many portions of the outer housing of the device as possible, since this gives a high-quality and solid impression to the user.

### TECHNICAL BACKGROUND

U.S. Pat. No. 10,765,148 B2 is related to an inhalation device, in which a housing and/or a button structure can be formed from a metal or ceramic. Also the press button shown in U.S. Pat. No. 10,412,785 B1 can be made of metal.

### SUMMARY OF THE INVENTION

Nevertheless, there remains a need for providing an inhalation device and a method of manufacturing the same which provide a high-quality impression of the device, combined with a simple internal structure.

This is, firstly, achieved by means of the subject-matter of claim 1.

Accordingly, the housing is at least partially made of a hard material and at least one button provided in an opening of the housing is at least partially made of a material substantially as hard as that of the housing or harder.

Further, the button is attached to the housing and/or the interior of the device by means of an elastic material inside of the housing.

By means of the latter feature, a relatively simple construction can be maintained, and the button, which has a relatively high hardness as such, is provided with the necessary flexibility, in order to allow pressing thereof so as to initiate a certain operation. The elastic material can for example be provided in the form of a sheet or thin plate, which can be adhered to the inner surface of the housing by means of an adhesive such as glue and/or by ultrasonic bonding.

Thus, the elastic material can be provided directly on the inside of the housing, but the button can, alternatively or additionally, also be attached to the interior of the device, such as a printed circuit board (PCB). Further, the button can have a step as described in more detail below, in order to prevent displacement from the device, and/or the button can be adhered to a portion of the elastic material. Further, a switch can be mounted on the PCB, to which the button is attached by means of the elastic material in such a manner that displacement of the button towards the inside of the device will operate the switch.

Either way described above ensures durability of the connection of the elastic material with the interior of the housing and, as a consequence, durability of the internal structure involving a relatively hard button. As mentioned above, such a button combined with a housing at least partially made of a hard material provides the impression of a high-quality and “genuine” product.

It should also be mentioned, that the device described herein differs from previously known devices in that the button is separated from the housing, unlike in common devices, in which the button is integrated in the housing and can be pushed by using the flexibility of commonly used, relatively soft plastic materials. As described herein, flexibility is achieved by means of elastic material attaching the button to the housing so as to make the button movable for activation by less than 1 mm, which can easily be guaranteed by means of the characteristics of the elastic material. In this context, the housing and/or the button and/or a plastic chassis described in more detail below can have a maximum thickness of about 0.8 mm, and the elastic material or sheet can have a maximum thickness of about 0.3 mm in those areas, which are adhered to the inside of the casing.

Advantageous embodiments are described in the further claims.

The above-described dimensions can advantageously be achieved, and the high-quality or premium impression of the product can be realized, when the housing and/or the button is made of a ceramic or metal material. Generally, using such hard materials also provides the advantage of durability of the device.

As regards the specific elastic material preferred, good properties are expected for an elastomer, in particular rubber and/or silicone.

The above-mentioned, hard materials also used for the housing do sometimes not inherently have the quality of allowing light to be transmitted therethrough. In order to make light from one or more light sources inside the device visible to the outside, the housing may have at least one locally thinned area.

A particularly simple and durable structure can be achieved for the device, if the housing has a removable front cover, in which at least one button as described above is provided.

Whereas the housing can at least partially be made of a relatively hard material, such as metal or ceramic, along its entire thickness, it provides economic advantages, when the housing at least partially has a chassis of plastic, to which the described material can be adhered so as to form the outside of the device. This will reduce the need for the amount of the described hard material, which is generally more expensive than plastic. The chassis can be attached to the housing before attaching the elastic material, and preferably also before attaching the button. All of the connections can be done by an adhesive, such as glue.

As regards the desired premium impression of the housing and/or the button, a hardness of at least HV19 and/or a maximum of HV2350 is currently preferred. The latter hardness corresponds to the use of ceramic material, and for the lower value it is still expected to provide the desired feel and impression to the user.

A simple and at the same type reliable structure can be realized, when the housing has at least one step, as seen in a sectional view thereof, preventing the button from extending beyond the outer surface of the housing. This will give a smooth and continuous impression of the outer surface and at the same time allow pressing of the button.

The above-mentioned object underlying the invention is further solved by means of the method described in claim 10. Corresponding to the device features, the method involves providing a housing at least partially made of a hard material and providing at least one button at least partially made of a material as hard as that of the housing or harder and attaching the at least one button in an opening of the housing by means of an elastic material attached to the inside of the housing and/or the interior of the device. Preferred features of the method described herein correspond to those of the described inhalation device and any features described with regard to the device and the method only, respectively, are likewise applicable to the method and the device, respectively.

As regards the order of manufacture, at least one chassis of plastic can be attached to at least a part of the housing before attaching the button.

Moreover, efficient manufacture can be insured by attaching the button to the elastic material, before the latter is attached to the housing. If a plastic chassis if present, the elastic material can be attached to the chassis, before attaching the button to the elastic material. The chassis then needs to have an opening large enough to allow attachment of the button from the outside, i.e. opposite the elastic material, and the housing having a smaller opening, large enough to accommodate the button, can then be attached to the chassis from outside.

Although the button can be cut from any piece of material, it provides advantages, if it is cut from a piece of material constituting the housing. In this manner, firstly, materials of the housing and the button fully correspond to each other. Secondly, when cutting the button, some material will be removed, which then automatically provides an appropriate gap between the button and the housing material, which allows pressing down movement of the button. Cutting can for example be done by a laser. When the housing and the button are provided separately, components can be moulded instead of cut.

### BRIEF DESCRIPTION OF THE DRAWINGS

Hereinafter, the invention will be described with reference to exemplary embodiments thereof and referring to the figures, in which

FIG. 1 shows a cut view of a cover forming a housing part of an inhalation device,

FIG. 2 shows a schematic sectional view of a second embodiment,

FIG. 3 shows a schematic sectional view of a third embodiment, and

FIG. 4 shows the embodiment of FIG. 3 with a pressed button.

### DETAILED DESCRIPTION OF PREFERRED EMBODIMENTS

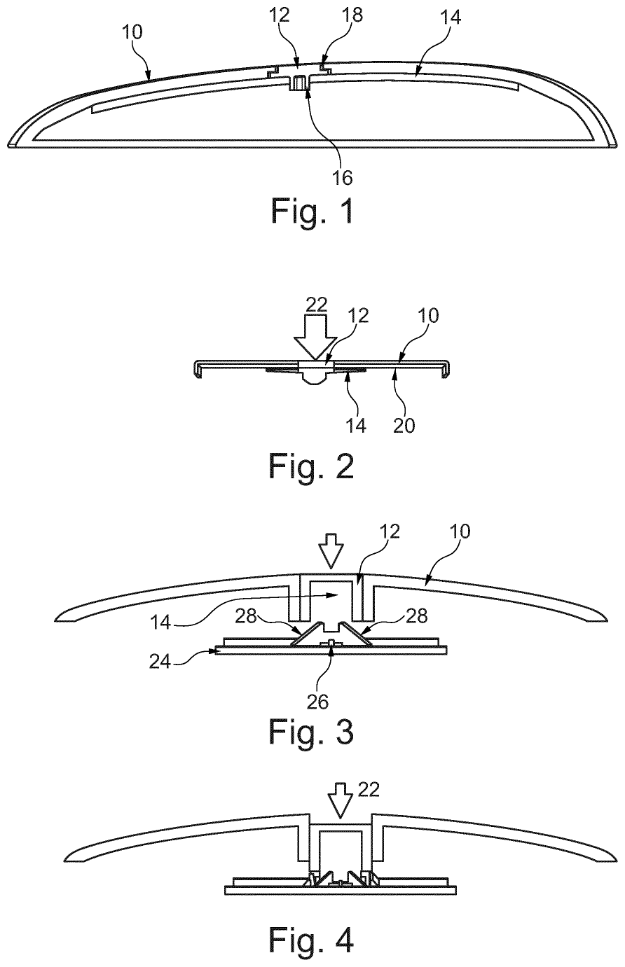

As can be taken from FIG. 1, a housing part, such as a cover 10 of an inhalation device can have a convex shape so as to create, due to an essentially constant thickness, a concave inside for accommodating the interior of the device. In the embodiment of FIG. 1, the cover 10 as well as a button 12 is made from ceramic, and the button 12 is separate from the cover 10, but attached to an elastic material in the form of a sheet 14, which is in turn attached to the interior of the cover 10. This essentially allows the button 12 to be pushed towards the interior of the device, so that a projection 16 provided on the inside of the button 12 can for example operate a switch.

As can be taken form the figure, the outside surfaces of the cover 10 and the button 12 are essentially continuous, so that a continuous and smooth outside of the device can be insured. This position is essentially defined by a step 18 provided on the button 12, which cooperates with a complementary step on the cover 10 to define the shown, outermost position of the button 12. As can be seen in the figure, the step can be provided on two opposing sides of the button 12, and it can also be provided all around the button 12. In the embodiment shown, the button 12 has, apart from the projection 16 and the step 18, also a constant thickness.

This also applies to the alternative embodiment schematically shown in FIG. 2. In this embodiment, a plastic chassis 20 is attached to the interior of the cover 10, so that the ceramic material of the cover 10 can be made thinner and costs can be reduced while maintaining a premium impression of the device. In the embodiment of FIG. 2, the elastic material 14 is attached to the chassis 20, and FIG. 2 shows a situation, in which pressure in the direction of arrow 22 is applied to the button 12 so as to move it slightly towards the inside of the device.

In the embodiment of FIG. 3 the button 12 does not comprise a step, but is provided in an opening of the cover 10 and is firmly attached to a solid portion of the elastic material 14. This material further comprises sheet-like portions which are in this case attached to a PCB 24, on which a switch 26 is provided underneath, or generally inside, referring to the device, the button. The solid portion of the elastic material 14, which is fully accommodated in the interior of the button 12 having essentially a cross-sectional shape of an inverted U, is connected to the sheet-like portions attached to the PCB 24 by means of relatively thin legs 28.

As can be taken from FIG. 4, when the button is pressed in direction of the arrow 22, the thin portions 28 collapse and thus allow operation of the switch 26 by means of the solid portion of the elastic material 14, or, alternatively, the button itself (not shown). It should be mentioned that the button can have any desired shape in a plan view, i.e. when viewed from the top of FIGS. 3 and 4, such as a circle, and also the sheet-like portion of the elastic material 14 can for example be circular when viewed from the top of FIGS. 3 and 4. Also, the legs 28 can be continuous all around so as to essentially form a cone, or they can be formed discontinuous. Moreover, all portions of the elastic material can be integrally formed in one piece of, for example, silicon.

1. An inhalation device with a housing at least partially made of a material and at least one button provided in an opening of the housing and at least partially made of a material substantially as hard as that of the housing or harder, wherein the at least one button is attached to the housing and/or an interior of the device by an elastic material inside of the housing.

2. The inhalation device of claim 1, wherein the material of the housing is a ceramic or metal material.

3. The inhalation device of claim 1, wherein the material of the at least one button is a ceramic or metal material.

4. The inhalation device of claim 3, wherein the elastic material is made of rubber and/or silicone.

5. The inhalation device of claim 1, wherein the housing has at least one locally thinned area configured to allow light transmission.

6. The inhalation device of claim 1, wherein the housing has a removable front cover, in which the at least one button is provided.

7. The inhalation device of claim 1, wherein the housing at least partially has a chassis of plastic.

8. The inhalation device of claim 1, wherein the housing and/or the at least one button has a hardness of at least HV19 and/or up to HV2350.

9. The inhalation device of claim 1, wherein the housing has at least one step preventing the at least one button from extending beyond an outer surface of the housing.

10. A method of producing an inhalation device including the steps of providing a housing at least partially made of a material and providing at least one button at least partially made of a material substantially as hard as that of the housing or harder, and attaching the at least one button in an opening of the housing by an elastic material attached to an inside of the housing and/or an interior of the device.

11. The method of claim 10, wherein at least one chassis of plastic is attached to at least a part of the housing before attaching the at least one button.

12. The method of claim 10, wherein the at least one button is attached to the elastic material beforehand.

13. The method of claim 10, wherein the housing is locally thinned, so as to allow light transmission.

14. The method of claim 10, wherein the at least one button is cut from a piece of material constituting the housing.

15. The method of claim 10, wherein the at least one button is prevented from extending beyond an outer surface of the housing by at least one step.
